(19) United States
(12) Patent Application Publication
(10) Pub. No.: US 2009/0030071 A1
(43) Pub. Date: Jan. 29, 2009

(54) Treatment of Cardiovascular Disease in Mexican Americans Using Nebivolol

(76) Inventors: Andrew A. Shaw, Morgantown, WV (US); R. Preston Mason, Manchester, MA (US); Betty Riggs, Morgan Town, WV (US)

Correspondence Address: FROST BROWN TODD, LLC, 2200 PNC CENTER, 201 E. FIFTH STREET, CINCINNATI, OH, 45202, US

(21) Appl. No.: 12/173,125

(22) Filed: Jul. 15, 2008

Related U.S. Application Data
(60) Provisional application No. 60/952,015, filed on Jul. 26, 2007.

(51) Int. Cl. A61K 31/352; A61P 9/00

(52) U.S. Cl. 514/456

(57) ABSTRACT

Nebivolol has been shown to be beneficial in the treatment of cardiovascular diseases such as, but not limited to, hypertension, congestive heart failure, arterial stiffness and endothelial dysfunction. The present invention relates to methods for treating and/or preventing cardiovascular disorders in persons of Hispanic descent, particularly Mexican Americans, using compositions comprising nebivolol.

### CROSS REFERENCE TO RELATED APPLICATIONS

The present application hereby claims the benefit of the provisional patent application of the same title, Ser. No. 60/952,015, filed on Jul. 26, 2007, the disclosure of which is hereby incorporated by reference in its entirety.

### BACKGROUND OF THE INVENTION

Treatment of cardiovascular diseases, particularly those associated with endothelial dysfunction, is a major health concern in the United States. Cardiovascular diseases associated with endothelial dysfunction include, but are not limited to, hypertension, diabetes, dylipidemia, heart failure, coronary artery disease, ischemic disease and atherosclerosis.

Hypertension, in particular, is a major health concern. Approximately 50 million Americans have elevated blood pressure defined as a systolic blood pressure (SBP)≧140 mmHG or a diastolic blood pressure (DBP) of 90 mmHG. Chobanian A V; Bakris G L; Henry R. Black; William C. Cushman; Lee A. Green; Joseph L. Izzo, Jr; Daniel W. Jones; Barry J. Materson; Suzanne Oparil; Jackson T. Wright, Jr; Edward J. Roccella; the National High Blood Pressure Education Program Coordinating Committee. The Seventh Report of the Joint National Committee on Prevention, Detection, Evaluation, and Treatment of High Blood Pressure: the JNC 7 report. JAMA. 289(19):2560-72, 2003. Epub 2003 May 14. Erratum in: JAMA. 2003 Jul. 9; 290(2):197.

In addition, individuals with blood pressure of 120/80 mmHg or higher are at increased risk of developing hypertension and are considered to be in a “prehypertensive” state. Vasan R S, Larson M G, Leip E P, Evans J C, O'Donnell C J, Kannel W B, Levy D. Impact of high-normal blood pressure on the risk of cardiovascular disease. N Engl J Med. 1; 345(18):1291-7, 2001; Vasan R S, Larson M G, Leip E P, Kannel W B, Levy D. Assessment of frequency of progression to hypertension in non-hypertensive participants in the Framingham Heart Study: a cohort study. Lancet. 17; 358(9294):1682-6, 2001.; Chobanian, 2003. Severity of hypertension is currently classified by stage, with Stage 1 hypertension spanning blood pressure ranges from 140/90 to 159/99 mmHg and Stage 2 including blood pressures ≧160/100 mmHg (Chobanian, 2003).

Onset of hypertension (diastolic alone or in combination with systolic) typically occurs between 25 and 55 years of age. The risk of developing hypertension increases more dramatically with increasing age. According to the National Health Interview Survey conducted in 1999, 44% of individuals between 65 and 74 years of age and 48% of those over 75 had been diagnosed as being hypertensive. Pleis J R, Coles R. Summary health statistics for U.S. adults: National Health Interview Survey, 1999. Vital Health Stat 10. (212):1-137, 2003.

The pathophysiology of hypertension is relatively unclear. About 95% of those diagnosed with hypertension have no clear single identifiable cause and are classified as patients with “essential hypertension.” It is more than likely that numerous interrelated factors contribute to the increase in blood pressure in hypertensive patients and their relative roles may differ between individuals. Factors that have been implicated in the pathogenesis of hypertension include: increased sympathetic tone; alterations in the renin-angiotensin system; endothelial dysfunction; diastolic dysfunction; hypercoagulability; insulin sensitivity; dietary factors, including increased salt intake; genetic factors; intrauterine influences; and neurovascular anomalies. Beevers D G, Lip G Y. The protective effect of blocking angiotensin in both type I and type II diabetics with nephropathy. J Hum Hypertens. 15(12):837-9, 2001. Review; Beevers D G. Salt and cardiovascular disease: not just hypertension. J Hum Hypertens. 15(11):749-50, 2001.

Hispanics are the largest and fastest-growing minority group in the United States, and Mexican Americans are the largest sub-group of Hispanics. Epidemiologic studies indicate that Mexican Americans have higher rates of coronary heart disease (CHD) risk equivalents, including type 2 diabetes mellitus, metabolic syndrome and some primary forms of dyslipidemia as compared to Non-Hispanic Whites.

Thus, pharmaceutical formulations for the treatment of cardiovascular diseases, especially formulations for the treatment of persons of Hispanic descent, particularly Mexican Americans, are needed.

### BRIEF SUMMARY OF THE INVENTION

In one aspect, the present invention relates to a method of reducing morbidity and mortality associated with cardiovascular disease in a person of Hispanic descent, particularly Mexican Americans, comprising administering to the patient a therapeutically effective amount of nebivolol, a metabolite of nebivolol, or a pharmaceutically acceptable salt thereof. The cardiovascular disease may be selected from the group consisting of congestive heart failure, hypertension, pulmonary hypertension, myocardial and cerebral infarctions, atherosclerosis, atherogenesis, thrombosis, ischemic heart disease, post-angioplasty restenosis, coronary artery diseases, renal failure, stable, unstable and variant (Prinzmetal) angina, cardiac edema, renal insufficiency, nephrotic edema, hepatic edema, stroke, transient ischemic attacks, cerebrovascular accidents, restenosis, controlling blood pressure in hypertension, platelet adhesion, platelet aggregation, smooth muscle cell proliferation, pulmonary edema, vascular complications associated with the use of medical devices, wounds associated with the use of medical devices, pulmonary thromboembolism, cerebral thromboembolism, thrombophlebitis, thrombocytopenia and bleeding disorders.

In one aspect, the invention relates to a method of reducing morbidity and mortality associated with cardiovascular disease in persons of Hispanic descent, particularly Mexican Americans, comprising administering to the patient nebivolol or pharmaceutically acceptable salt thereof in an amount of from about 0.125 mg per day to about 40 mg per day.

In another aspect, the invention relates to a method of treating or preventing a cardiovascular disorder in a person of Hispanic descent, particularly Mexican Americans, in need thereof, comprising administering to the person of a safe and therapeutically effective amount of nebivolol or a pharmaceutically acceptable salt thereof.

In yet another aspect, the invention relates to a method for improving NO release in persons of Hispanic descent, particularly Mexican Americans, in need thereof, comprising administering to the person of Hispanic descent a safe and therapeutically effective amount of nebivolol or a pharmaceutically acceptable salt thereof.

In yet another aspect, the present invention relates to a method for improving exercise tolerance or for improving the quality of life in a persons of Hispanic descent, particularly Mexican Americans, in need thereof comprising administering to the patient a therapeutically effective amount of nebivolol or a pharmaceutically acceptable salt thereof.

In yet another embodiment, the present invention relates to a method of increasing endothelial nitric oxide production in persons of Hispanic descent, particularly Mexican Americans, comprising administering to the person nebivolol or a pharmaceutically acceptable salt in a therapeutically effective amount.

In yet another embodiment, any of the above methods can be used wherein nebivolol or a pharmaceutically acceptable salt thereof is administered in combination with a cardiovascular agent selected from the group consisting of ACE inhibitors, ARB's, adrenergic blockers, adrenergic agonists, agents for pheochromocytoma, anti-arrhythmics, anti-platelet agents, anticoagulants, anti-hypertensives, anti-lipemic agents, anti-diabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, rennin inhibitors, vasodilators, vasopressors, cholesterol or lipid lowering agents such as, for example, statins, AGE crosslink breakers, AGE formation inhibitors, and mixtures thereof.

These embodiments of the present invention, other embodiments, and their features and characteristics, will be apparent from the description, drawings and claims that follow.

### BRIEF DESCRIPTION OF THE DRAWING

FIG. 1 depicts a comparison of NO release from non-Hispanic white (open bars) and Mexican American (solid bars) donor HUVECs after acute treatment with nebivolol at 1 μM, 3 μM and 5 μM concentrations. * p<0.05 versus non-Hispanic whites at cognate drug concentration (Student t-test).

FIG. 2 depicts a comparison of NO release following acute stimulation with various nebivolol concentrations from non-Hispanic white (open bars) and Mexican American (solid bars) donors following a pre-incubation for 24 hours with nebivolol at 1.0 μM. Values are mean±S.E. (n=6). **p<0.001 and *p<0.05 versus non-Hispanic white at cognate drug concentration (Student t-test).

FIG. 3 depicts a comparison of NO release following acute stimulation with various nebivolol concentrations from non-Hispanic white (open bars) and Mexican American (solid bars) donors following a pre-incubation for 24 hours with nebivolol at 5.0 μM. Values are mean±S.E. (n=6). ***p<0.0001, **p<0.01 and *p<0.05 versus non-Hispanic white at cognate drug concentration (Student t-test).

FIG. 4 depicts a comparison of the change in endothelial-dependent NO release from non-Hispanic white (open bars) and Mexican American (solid bars) donors with acute stimulation with either 1.0 μM or 5.0 μM nebivolol or atenolol following pre-incubation for 24 hours with either nebivolol or atenolol at 1.0 μM. Values are mean±S.E. (n=6). **p<0.0001 and *p<0.05 versus non-Hispanic white at cognate drug concentration (Student t-test.)

FIG. 5 depicts a comparison of the change in endothelial-dependent NO release from non-Hispanic white (open bars) and Mexican American (solid bars) donors with acute stimulation with either 1.0 μM or 5.0 μM nebivolol or atenolol following pre-incubation for 24 h with either nebivolol or atenolol at 5.0 μM. Values are mean±S.E. (n=6). **p<0.0001 and *p<0.01 versus non-Hispanic white at cognate drug concentration (Student t-test).

FIG. 6 depicts the effects of STZ-induced diabetes on the ratio of CaI-induced NO/ONOO release from aortic endothelial cells in the absence and presence of nebivolol treatment prior to sacrifice of animals at 13 weeks. Values are reported as mean±SD. *p<0.0001 versus control and ^{†}p<0.001 versus STZ treatment (Student t-test).

FIG. 7 depicts the effects of STZ-induced diabetes on CaI-induced endothelial-dependent NO release from aortic endothelial cells in the absence and presence of nebivolol treatment prior to sacrifice of animals at 13 weeks. Values are reported as mean±SD. *p<0.0001 versus control and ^{†}p<0.001 versus STZ treatment (Student t-test). After treatment with nebivolol, there was not a statistically significant difference as compared to non-diabetic control animals (p=0.1273).

FIG. 8 shows the potentiating effects of nebivolol on israpidine. Panel A graphs the percent increase in CaI-induced NO release for the sum of nebivolol and isradipine separate effects versus the combined effects of nebivolol and isradipine at 1.0 μM in endothelial cells from black American downers, whereas Panel B depicts those effects in endothelial cells from white American doners. The increase in NO release from the combination was greater than the sum of their separate effects. Values are mean±S.D. (n=6).

FIG. 9 shows the potentiating effects of nebivolol on verapamil. Panel A graphs the percent increase in CaI-induced NO release for the sum of nebivolol and verapamil separate effects versus the combined effects of nebivolol and verapamil at 1.0 μM in endothelial cells from black American downers, whereas Panel B depicts those effects in endothelial cells from white American doners. The increase in NO release from the combination was greater than the sum of their separate effects. Values are mean±S.D. (n=6).

FIG. 10 shows the potentiating effects of nebivolol on diltiazem. Panel A graphs the percent increase in CaI-induced NO release for the sum of nebivolol and diltiazem separate effects versus the combined effects of nebivolol and diltiazem at 1.0 μM in endothelial cells from black American downers, whereas Panel B depicts those effects in endothelial cells from white American donors. The increase in NO release from the combination was greater than the sum of their separate effects. Values are mean±S.D. (n=6).

### DETAILED DESCRIPTION OF THE INVENTION

### Definitions

For convenience, certain terms employed in the specification, examples and claims are collected here. These definitions should be read in light of the remainder of the disclosure and understood as by a person of skill in the art. Unless defined otherwise, all technical and scientific terms used herein have the same meaning as commonly understood by a person of ordinary skill in the art. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention. In order to provide a clear and consistent understanding of the specification and claims, including the scope to be given such terms, the following definitions are provided:

The articles “a” and “an” are used herein to refer to one or to more than one (i.e., to at least one) of the grammatical object of the article. By way of example, “an element” means one element or more than one element.

The term “antagonist” is art-recognized and refers to a compound that binds to a receptor site, but does not cause a physiological change unless another receptor ligand is present.

The term “bioavailable” is art-recognized and refers to a form of the subject invention that allows for it, or a portion of the amount administered, to be absorbed by, incorporated to, or otherwise physiologically available to a subject or patient to whom it is administered.

The phrase “cardiovascular agent” or “cardiovascular drug” refers to a therapeutic compound that is useful for treating or preventing a cardiovascular disease. Non-limiting examples of suitable cardiovascular agents include ACE inhibitors (angiotensin II converting enzyme inhibitors), ARB's (angiotensin II receptor antagonists), adrenergic blockers, adrenergic agonists, agents for pheochromocytoma, antianginal agents, antiarrhythmics, antiplatelet agents, anticoagulants, antihypertensives, antilipemic agents, antidiabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, rennin inhibitors, vasodialators, vasopressors, AGE crosslink breakers (advanced glycosylation end-product crosslink breakers, such as alagebrium, see U.S. Pat. No. 6,458,819), and AGE formation inhibitors (advanced glycosylation end-product formation inhibitors, such as pimagedine), and combinations thereof.

Cardiovascular disease or disorder refers to any cardiovascular disease or disorder known in the art, including, but not limited to, those selected from the group consisting of congestive heart failure, hypertension, pulmonary hypertension, myocardial and cerebral infarctions, atherosclerosis, atherogenesis, thrombosis, ischemic heart disease, post-angioplasty restenosis, coronary artery diseases, renal failure, stable, unstable and variant (Prinzmetal) angina, cardiac edema, renal insufficiency, nephrotic edema, hepatic edema, stroke, transient ischemic attacks, cerebrovascular accidents, restenosis, controlling blood pressure in hypertension, platelet adhesion, platelet aggregation, smooth muscle cell proliferation, pulmonary edema, and vascular complications associated with the use of medical devices.

The term “combination” refers to two or more different active agents that are administered at roughly about the same time (for example, where the active agents are in a single pharmaceutical preparation) or at different times (for example, one agent is administered to the subject before the other).

The terms “drug,” “pharmaceutically active agent,” “bioactive agent,” “therapeutic agent,” and “active agent” may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

The phrase “effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

“Endothelial dysfunction” refers to the impaired ability of in any physiological processes carried out by the endothelium, in particular, production of nitric oxide regardless of cause. It may be evaluated by, such as, for example, invasive techniques, such as, for example, coronary artery reactivity to acetylcholine or methacholine, and the like, or by noninvasive techniques, such as, for example, blood flow measurements, brachial artery flow dilation using cuff occlusion of the arm above or below the elbow, brachial artery ultrasonography, imaging techniques, measurement of circulating biomarkers, such as, asymmetric dimethylarginine (ADMA), and the like. For the latter measurement the endothelial-dependent flow-mediated dilation will be lower in patients diagnosed with an endothelial dysfunction.

“Hispanics” or “Latinos” are those people who classify themselves in one of the specific Spanish, Hispanic, or Latino categories listed on the Census 2000 questionnaire—“Mexican, Mexican Am., Chicano,” “Puerto Rican,” or “Cuban”—as well as those who indicate that they are “other Spanish/Hispanic/Latino.” Persons who indicated that they are “other Spanish/Hispanic/Latino” include those whose origins are from Spain, the Spanish-speaking countries of Central or South America, the Dominican Republic or people identifying themselves generally as Spanish, Spanish-American, Hispanic, Hispano, Latino, and so on. Origin can be viewed as the heritage, nationality group, lineage, or country of birth of the person or the person's parents or ancestors before their arrival in the United States. People who identify their origin as Spanish, Hispanic, or Latino may be of any race. In general, “Hispanic” refers to a person of Mexican, Puerto Rican, Cuban, Central or South American, or other Spanish culture or origin, including Mexican-Americans and Hispanics of Caribbean descent.

“Non-Hispanic White” persons are those who respond “No, not Spanish/Hispanic/Latino” and who report “White” as their only entry in a race question.

The phrase “nitric oxide synthase” or “NOS” refers to enzymes that produce nitric oxide. The phrase “endothelial nitric oxide synthase” or “eNOS” refers to the predominant form of the enzyme that produces nitric oxide in the vasculature. The phrase includes, without limitation, all forms of the enzyme, including those proteins expressed from sequence having polymorphisms, deletions, mutations, truncations, and/or substitutions of nucleic acids, and all forms of the enzyme, including forms having deletions, mutations, truncations or substitutions of amino acids of the enzyme, known or unknown. As used herein, “modulation” of eNOS activity is used in a broad sense and refers to the ability to induce or enhance, inhibit or decrease, or maintain eNOS protein expression and/or activity.

The phrase “nebivolol composition” refers to a composition comprising nebivolol. Nebivolol is a mixture of d and l isomers of α, α′-[iminobismethylene]bis[6-fluoro-3,4-dihydro-2H-1-benzopyran-2-methanol]. The composition may include at least one other cardiovascular agent or at least one pharmaceutically acceptable carrier or both.

A “patient,” “subject” or “host” may be a human or non-human animal.

Pathologic conditions and disorders “associated with” NO or “characterized by” eNOS dysfunction and/or wherein NO is an “important regulator” are those conditions and disorders where nitric oxide insufficiency or excess is correlated with disease. Such disorders and conditions include, for example, hypertension, diabetes, thrombosis, angina, atherosclerosis, and heart failure, wherein nitric oxide levels in mammalian cells or tissues are present in insufficient quantities as compared to normal or healthy mammalian cells or tissues. Further applications in which the use of nebivolol as described herein may be beneficial include the use of nebivolol to increase eNOS or NO production prior to, concurrent with or subsequent to angioplasty to prevent restenosis or neointima formation.

The term “pharmaceutically acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be acceptable in the sense of being compatible with the subject composition and its components and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) IV fluids, including but not limited to Ringer's solution, 5% dextrose in water, and half normal saline; (19) ethyl alcohol; (20) phosphate buffer solutions; and (21) other non-toxic compatible substances employed in pharmaceutical formulations, such as, for example, eudrigits and waxes such as carnuba wax.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of any condition or disease.

Hispanics are the largest and fastest-growing minority group in the United States, and Mexican Americans are the largest sub-group of Hispanics. Epidemiologic studies indicate that Mexican Americans have higher rates of coronary heart disease (CHD) risk equivalents, including type 2 diabetes mellitus, metabolic syndrome and some primary forms of dyslipidemia as compared to Non-Hispanic Whites Stern et al., Diabetes Care 14: 649-654, 1991; Aguilar-Salinas C, Olaiz G, Valles V, Torres J M, Gomez-Perez F J, Rull J A, Rojas R, Franco A, Sepulveda J. High prevalence of low HDL cholesterol concentrations and mixed hyperlipidemia in a Mexican nationwide survey. J Lipid Res. 42: 1298-1307, 2001; Aguilas-Salinas C, Velazquez-Monroy O, Gomez-Perez F J, Gonzalez-Chavez A, Esqueda A L, Molina-Cuevas V, Rull-Rodrigo J A, Tapia-Conyer R. for the ENSA 2000 Group. Characteristics of patients with type 2 diabetes in Mexico: Results from a large population-based nationwide survey. Diabetes Care. 26: 2021-2026, 2003. By the age of 50, epidemiologic studies indicate that 28% of men and 21% of women in Mexico already exhibit some form of dyslipidemia. Aguilar-Salina et al., 2001. In the San Antonio Heart Study, The age- and sex-adjusted hazard ratio (HR) for all-cause mortality comparing U.S.-born Mexican Americans (MAs) with Non-Hispanic Whites (NHWs) was 1.7 (95% CI. 1.5-2.40), while comparing Mexico-born MAs with NHWs was 1.14 (95% CI 0.63-2.06). Hunt K J, Williams K, Resendez R G, Hazuda H P, Haffner S M, Stern M P. All-cause and cardiovascular mortality among diabetic participants in the San Antonio heart study: Evidence against the “Hispanic Pradox”. Diabetes Care. 26: 1557-1563, 2002. For CVD, U.S.-born Mexican Americans were 1.7 times more likely to die from CVD compared to NHWs. Hunt et al., 2002. There are also well-known environmental factors that contribute to higher risk in Mexican Americans, including a high-fat and high caloric diet, tobacco use, alcohol consumption and sedentary lifestyle.

Hypertension is a risk factor that is less likely treated and controlled among Hispanics, as compared to the overall U.S. population. This was a key finding from the National Health and Nutrition Examination Surveys (NHANES) for 1999-2002. This report has identified racial/ethnic disparities in the awareness of, treatment for, and control of hypertension. NHANES is a stratified, multistage probability sample of the civilian, non-institutionalized U.S. population. Both the survey interview population of 7,000 U.S. adults aged >20 years and the 5,000 respondents who completed the health examination each year included low-income persons, persons aged >60 years, blacks, and Mexican Americans. The analysis is based on data from non-Hispanic whites, non-Hispanic blacks, or Mexican Americans with BP measurements. Pregnant women were excluded from the analysis. For this analysis, hypertension was defined as having an average systolic BP>140 mm Hg or diastolic BP>90 mm Hg or taking BP medication. BP measures were based on the average of three BP readings. Persons with hypertension were considered 1) to be aware of their condition if they reported in the interview that a health-care professional had told them their BP was high, 2) to have been treated if they reported using antihypertensive medication, and 3) to have controlled BP if they were hypertensive but their BP measurements were <140/90 mm Hg. Statistical software was used to obtain weighted population estimates, age-specific and age-standardized prevalences and proportions, and 95% confidence intervals (CI).

During 1999-2002, the age-adjusted prevalence of hypertension in the study population was 28.6% (CI=26.8%-30.4%). The prevalence of hypertension increased with age, as expected, and was higher among women than men. Among adults with hypertension, the proportion aware of having this condition was 70.3% among non-Hispanic blacks, 62.9% among non-Hispanic whites, but only 49.8% among Mexican Americans. The age-adjusted proportion who reported treatment was 55.4% among non-Hispanic blacks, 48.6% among non-Hispanic whites, and again only 34.9% among Mexican Americans. Only 29% of U.S. adults with hypertension had controlled BP levels (<140/90 mm Hg). The proportion with controlled BP was similar among non-Hispanic blacks (29.8%) and non-Hispanic whites (29.8%) but substantially lower among Mexican Americans (17.3%). These findings indicate the challenge of effectively treating and controlling hypertension in the rapidly growing Hispanic and Mexican American population.

Hispanics also have a greater incidence of diabetes than non-Hispanic whites. Stern M P and Mitchell B D. Diabetes in Hispanic Americans. Diabetes in America, 2^{nd} Edition. Harris M I et al Editors. Bethesda, Md.: National Institutes of Diabetes and Digestive and Kidney Diseases, 1995. More specifically, Mexican-Americans have a two-fold greater chance of developing diabetes at some time in their life than non-Hispanic whites. Stern 1995. Since diabetes is a strong risk factor for development of cardiovascular disease, prevention of the damaging inflammatory process of diabetes within coronary vessels becomes vital. Levels of endothelial dysfunction serum marker (endothelin-1, Willebrand factor, and C-reactive protein) in patient's having type I diabetes mellitus begin to increase as their disease progresses from the microalbuminuria stage to the congestive renal failure stage. Shestakova M V, Yarek-Martynova I R, Kukharenko S S, Aleksandrov AnA, Dedov I I. Cardiorenal pathology in type 1 diabetes mellitus: Mechanisms of development and possibilities of medical correction. Terapevtichesky Arkhiv (Therapeutical Archives) 6: 40-45, 2005. This progression is marked with the activation of the inflammatory processes responsible for the development of atherosclerotic vessel damage, which as the disease progresses, will increase the patient's risk of developing some type of cardiovascular disease. Shestakova 2005. Cardiovascular diseases, in general, have been associated with reduced production of nitric oxide. Nitric oxide (NO) is an important protective molecule formed from L-arginine in the vasculature by NO synthase. Luscher T F. Imbalance of endothelium-derived relaxing and contracting factors. A new concept in hypertension? Am J Hypertens. 3(4):317-30, 1990.

Stimulation of intact endothelial cells by neurotransmitters, hormones, shear stress, and substances derived from platelets and the coagulation system causes release of NO derived from the endothelium. NO induces relaxation of the underlying vascular smooth muscle. Endothelial NO synthase (eNOS) is responsible for most of the NO produced in the tissue.

The endothelium modulates vascular tone through release of NO, via vasodilation that regulates regional blood flow. Endothelial dependent nitric oxide is responsible for maintenance of vascular tone, prevention of monocyte and platelet adhesion, decreases platelet aggregation, and decreases smooth muscle cell contraction. John S and Schmieder R E. Impaired endothelial function in arterial hypertension and hypercholesterolemia: potential mechanisms and differences. J Hypertens 18 (4): 363-374, 2000. A reduction in NO bioavailability contributes to elevated vascular resistance and loss of sensitivity to stimuli of vasodilation, hallmark features of hypertension. Beyond vasodilation, NO has additional, well-characterized vascular benefits, including inhibition of smooth muscle cell proliferation and migration, blocking adhesion of leukocytes to the endothelium, and preventing platelet aggregation.

Because vascular endothelial release of NO results in vasodilation, stimulation of its release, production/synthesis, or its ability to activate smooth muscle cell relaxation would have to be reduced and/or its rate of degradation increased for it to have a pathogenic role in the development of hypertension. Benjamin N, Vane J. Nitric oxide and Hypertension. Circulation. 15; 94(6):1197-8, 1996. Although a definitive role for deficient NO production in human essential hypertension remains to be established, the following observations support the possibility of such a relationship:

First, patients with hypertension and no overt atherosclerosis have endothelial dysfunction as documented by diminished reactive hyperemic flow (endothelium-dependent) in the brachial artery. Iiyama K, Nagano M, Yo Y, Nagano N, Kamide K, Higaki J, Mikami H, Ogihara T., Impaired endothelial function with essential hypertension assessed by ultrasonography. Am Heart J. 132(4):779-82, 1996. In contrast, the response to isosorbide dinitrate, which acts directly on smooth muscle (endothelium-independent), is unaltered. This suggests that the ability of NO to activate smooth muscle relaxation is not affected.

Further, offspring of hypertensive patients have a reduced vasodilator response to acetylcholine (which acts via a receptor-mediated release of NO) but not nitroprusside (which is endothelium-independent). Taddei S, Virdis A, Mattei P, Ghiadoni L, Sudano I, Salvetti A. Defective L-arginine-nitric oxide pathway in offspring of essential hypertensive patients. Circulation. 94:1298-1303, 1996. This suggests an alteration in the stimulation of NO release or production.

Finally, endothelium-dependent vasodilation, which is related to availability of L-arginine in normotensive subjects, was not influenced by increased substrate availability in patients with hypertension. Panza J A, Casino P R, Badar D M, Quyyumi A A. Effect of increased availability of endothelium-derived nitric oxide precursor on endothelium-dependent vascular relaxation in normal subjects and in patients with essential hypertension. Circulation. 87(5):1475-81, 1993.

In addition to its role in vasodilation, NO is a potent inhibitor of vascular smooth muscle cell migration and proliferation. Garg U C, Hassid A. Nitric oxide-generating vasodilators and 8-bromo-cyclic guanosine monophosphate inhibit mitogenesis and proliferation of cultured rat vascular smooth muscle cells. J Clin Invest. 83(5):1774-7, 1989. It also inhibits adhesion of platelets and white cells to the endothelium and in experimental models it protects against atherogenesis. Luscher, 1990; Thakur N K, Hayashi T, Sumi D, Kano H, Matsui-Hirai H, Tsunekawa T, Iguchi A. Anti-atherosclerotic effect of beta-blocker with nitric oxide-releasing action on the severe atherosclerosis. J Cardiovasc Pharmacol. 39(2):298-309, 2002. Moreover, NO appears to stimulate the adaptive mechanisms for reducing shear wall stress associated with chronically elevated arterial blood flow. Tronc F, Mallat Z, Lehoux S, Wassef M, Esposito B, Tedgui A. Role of matrix metalloproteinases in blood flow-induced arterial enlargement: interaction with NO. Arterioscler Thromb Vasc Biol. 20(12):E120-6, 2000. A marked loss in NO production by the endothelium results in: 1) increased vasoconstriction and vasospasm, 2) greater monocyte and low density lipoprotein infiltration, 3) proliferation of vascular smooth muscle cells, 4) increased oxidative stress, and 5) increased platelet aggregation. Anderson, T J. Nitric oxide, atherosclerosis and the clinical relevance of endothelial dysfunction. Heart Fail Rev. 8(1):71-86, 2003.

Abnormalities in endothelial function are also present in patients with coronary artery disease, diabetes and/or hyperlipidemias as well as in patients who smoke cigarettes. Vogel R A. Coronary risk factors, endothelial function, and atherosclerosis: a review. Clin Cardiol. 20(5):426-32, 1997; Celermajer D S, Sorensen K E, Gooch V M, Spiegelhalter D J, Miller O I, Sullivan I D, Lloyd J K, Deanfield J E. Non-invasive detection of endothelial dysfunction in children and adults at risk of atherosclerosis. Lancet. 340(8828):1111-5, 1992. It appears that endothelial function is a common denominator for all of the major cardiovascular risk factors. This raises the strong possibility that endothelial dysfunction is a significant factor in the development and/or progression of atherosclerotic coronary disease. Faxon et al., Circulation 109; 2595-2604, 2004.

While epidemiologic studies indicate a higher risk of CVD and lower BP control among Mexican Americans, the underlying pathophysiology is not well understood. Studies in other high-risk population, such as African Americans, indicate that the higher risk is related to reduced responsiveness of conductance vessels to both endogenous and exogenous stimulants of NO, as compared with age-matched whites. Campia U, Choucair W K, Bryant M B, Waclawiw M A, Cardillo C, Panza J A. Reduced endothelium-dependent and -independent dilation of conductance arteries in African Americans. J Am Col of Cardiol. 40:754-760, 2002; Kalinowski L, Dobrucki I T, Malinski T. Race-specific differences in endothelial function: Predisposition of African Americans to vascular disease. Circulation 109: 2511-2517, 2004. To understand the basis for this difference, it was reported that there is a lower NO bioavailability from endothelium of black Americans, despite much higher levels of endothelial-dependent NO synthase (eNOS). Kalinowski, 2004. The cellular basis for this paradox was the finding that excessive superoxide O₂ generation by NAD(P)H-oxidase and uncoupled eNOS resulted in the loss of functional NO due to its reactivity with O₂⁻, resulting in peroxynitrite (ONOO⁻) formation, a potent oxidant. Kalinowski, 2004.

We have now shown with in-vitro experimentation using human umbilical vein endothelial cells (HUVECs) isolated from Mexican American and non-Hispanic White female donors that Mexican Americans do have a statistically significant inherent reduction (approximately 43%) in their vein endothelial cells capacity to release nitric oxide (NO). This inherent loss of nitric oxide potentially predisposes Mexican-Americans to endothelial dysfunction, which has been associated with hypertension, atherosclerosis, vascular smooth muscle cell proliferation, platelet adhesion, and diabetes mellitus. Drexler H, Hayoz D, Munzel T, Hornig B, Just H, Brunner H R, Zelis R. Endothelial function in chronic congestive heart failure. Am J Cardiol. 69:1596-1601, 1992; Gilligan D M, Panza J A, Kilcoyne C M, Waclawiw M S, Casion P R, Quyyumi A A. Contribution of endothelium-derived nitric oxide to exercise-induced vasodilation. Circulation. 90:2853-2858, 1994.

Thus, agents that directly stimulate NO release may have important therapeutic advantages in the prevention and treatment of cardiovascular disease. Studies in other high-risk populations, such as African Americans, indicate that the higher CVD risk is related to reduced responsiveness of conductance vessels to both endogenous and exogenous stimulants of NO, as compared with age-matched whites. Campia et al., 2002, Kalinowski et al, 2004.

### Nebivolol

Nebivolol is described in U.S. Pat. No. 6,545,040, EP-0,145,067 and EP-0,334,429. EP-0,145,067 generally describes 2,2′-iminobisethanol derivatives useful for the treatment and/or prevention of disorders of the coronary vascular system. EP-0,334,429 describes [iminobismethylene]bis[3,4-dihydro-2H-1-benzopyran-2-methanol]derivatives including nebivolol. Nebivolol may be prepared according to the procedures described in EP-0,145,067 and more specifically in EP-0,334,429. Nebivolol is also described, for example, in U.S. Pat. No. 5,759,580, Jans et al.; U.S. Pat. No. 5,874,461, de Chaffoy de Courcelles, et al.; U.S. Pat. No. 6,075,046, de Chaffoy de Courcelles, et al.; and U.S. Pat. No. 6,545,040, Xhonneux, et al.

Nebivolol has basic properties and may be converted into its pharmaceutically acceptable acid addition salt forms by treatment with appropriate acids. Appropriate acids are, for example, inorganic acids, such as hydrohalic acid, e.g. hydrochloric, hydrobromic and the like, and sulfuric acid, nitric acid, phosphoric acid; or organic acids, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. The acid addition salt that is preferred in this invention is the hydrochloride acid addition salt. Nebivolol, via activation of an L-arginine/nitric oxide pathway and anti-oxidant properties, is an endothelial-dependent vasodilatory agent with cardioselective (β₁-selective) adrenoreceptor blocking capacity and direct vasodilatory properties. The mechanism of vasodilation is attributed to stimulation of endothelial-dependent NO synthase, an effect that can be completely inhibited with NG-monomethyl-L-arginine (L-NMMA), a nitric oxide synthase inhibitor. Of its two enantiomers, d-nebivolol possesses P i-adrenergic selectivity, while l-nebivolol lacks β₁-adrenergic activity. However, both enantiomers stimulate endothelial-dependent NO release, with evidence for greater activity with l-nebivolol, despite its absence of high β₁-selectivity. In cellular and animal models, nebivolol has been demonstrated to effect vasodilation through endothelial β₂-adrenergic receptor-mediated NO production and/or ATP efflux with consequent stimulation of P2Y-purinoceptor-mediated NO release. It has also been reported that nebivolol inhibits NO synthase uncoupling and produces systemic antioxidant effects.

Nebivolol is a β-receptor blocking drug that is a mixture of d- and l-enantiomers, of which d-nebivolol is a highly selective β₁-receptor antagonist.

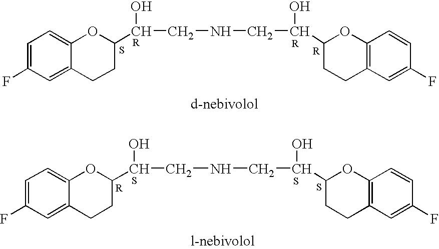

In addition to its β-receptor blocking properties, nebivolol has been shown to cause endothelium-dependent vasodilation in both normotensive and hypertensive subjects. Cockcroft J R, Chowienczyk P J, Brett S E, Chen C P, Dupont A G, Nueten L V, Wooding S J, Ritter J M., Journal of Pharmacology and Experimental Therapeutics. 1995; 274:1067-1071; Tzemos N, Lim P O, MacDonald T M., Circulation, 2001; 104:511-514; Broeders M A, Doevendans P A, Bekkers B C, Bronsaer R, van Gorsel E, Heemskerk J W, Egbrink M G, van Breda E, Reneman R S, van Der Zee R., Circulation 2000; 102:677-684. Bowman, A. J., CPL-H Chen, G A Ford. Br. J. Clin. Pharmac. 1994; 38:199-204. In experimental models, nebivolol has been demonstrated to stimulate NO release through β₂-adrenergic receptor-mediated NO production and/or ATP efflux with consequent stimulation of P2Y-purinoceptor-mediated NO release. Broeders M A, Doevendans P A, Bekkers B C, Bronsaer R, van Gorsel E, Heemskerk J W, Egbrink M G, van Breda E, Reneman R S, van Der Zee R., Circulation, 2000; 102:677-684; Kalinowski L, Dobrucki L W, Szczepanska-Konkel M, Jankowski M, Martyniec L, Angielski S, Malinski T., Circulation, 2003; 107:2747-2752. It has also been reported that nebivolol inhibits NO synthase uncoupling and produces systemic antioxidant effects. Mollnau H, Schulz E, Daiber A, Baldus S, Oelze M, August M, Wendt M, Walter U, Geiger C, Agrawal R, Kleschyov A L, Meinertz T, Thomas Munzel T., Arteriosclerosis, Thrombosis, and Vascular Biology. 2003; 23:615-621; Troost R, Schwedhelm E, Rojczyk S, Tsikas D, Frolich J C., British Journal of Clinical Pharmacology, 2000; 50:377-379.

As set forth above, the underlying pathophysiology of CVD and lower BP control among Mexican Americans is not well understood, though studies in other high-risk populations indicate that reduced responsiveness of conductance vessels to both endogenous and exogenous stimulants of NO may be a contributing factor. (Campia et al, 2002, Kalinowski et al, 2004). We have now shown with in-vitro experimentation using human umbilical vein endothelial cells (HUVECs) isolated from Mexican-American and non-Hispanic White female donors that Mexican-Americans have a statistically significant inherent (approximately 43%) reduction in the capacity of their vein endothelial cells to release nitric oxide (NO) relative to non-Hispanic Whites. This inherent loss of nitric oxide potentially predisposes Mexican-Americans to endothelial dysfunction, which has been associated with hypertension, atherosclerosis, vascular smooth muscle cell proliferation, platelet adhesion, and diabetes mellitus (Drexler, 1992, Gilligan, 1994).

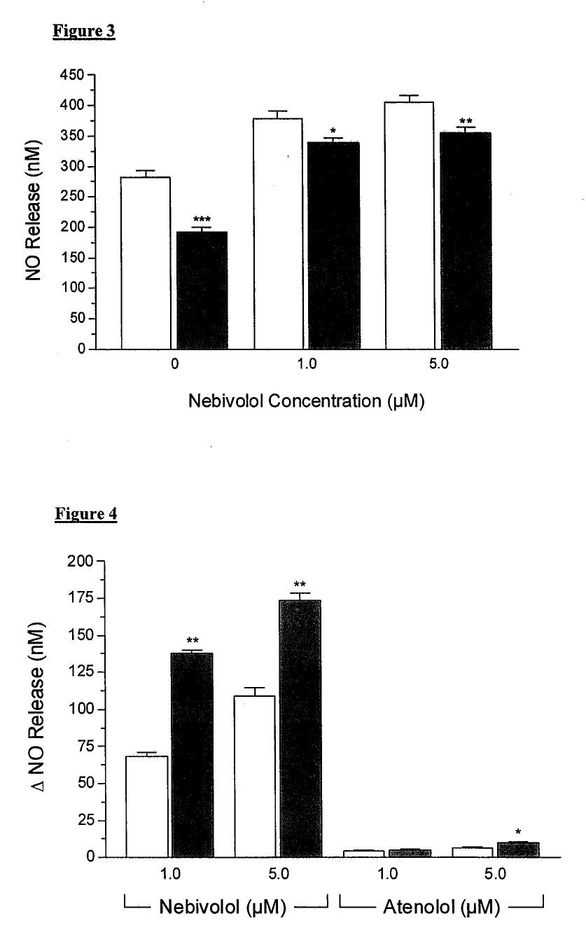

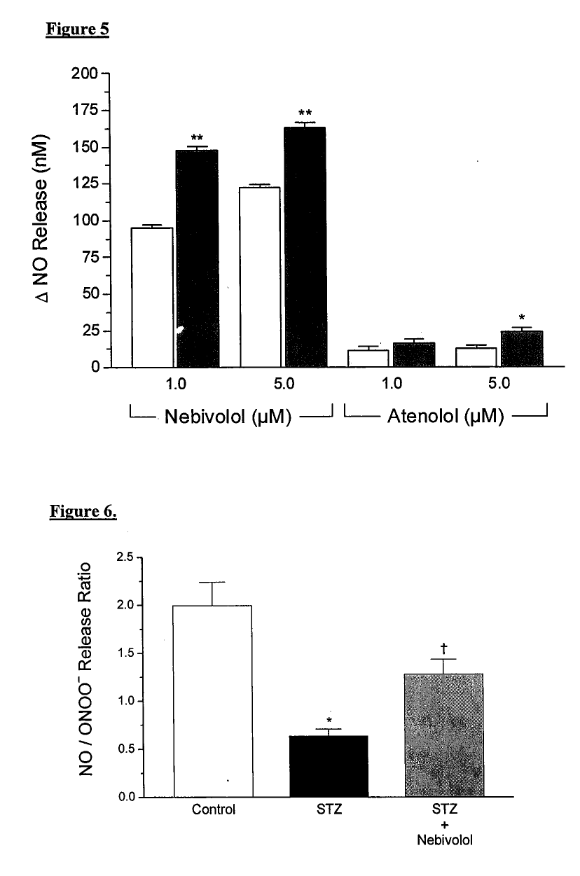

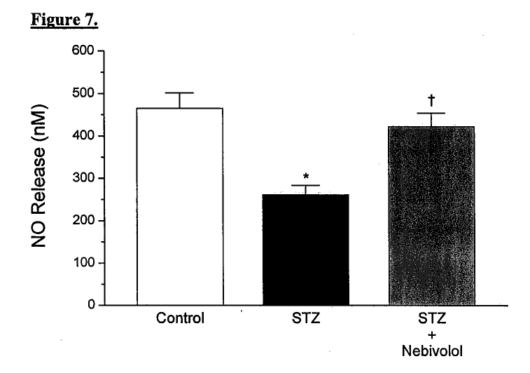

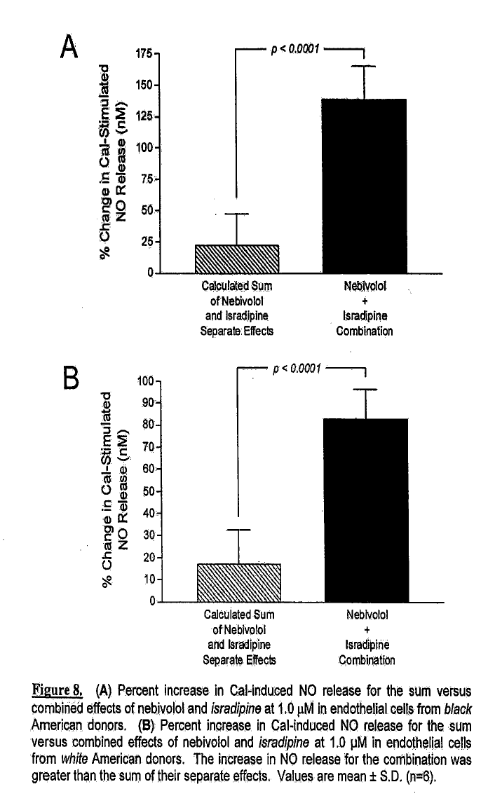

A statistically significant and beneficial effect from nebivolol in the Mexican-American HUVECs was observed over the drug treatment range of about 1 to about 5 μM, though beneficial effects may be observed at doses as low as 0.01 μM. Following a 24-hour pre-incubation of HUVEC cells with nebivolol at two different concentrations (1.0 and 5.0 μM), we have now shown that cells from the Mexican-American donors demonstrate a greater increase in NO bioavailability relative to non-Hispanic White counterparts (see FIGS. 4 and 5). This increase in NO production is not due to a beta-adrenergic receptor blockade effect, as atenolol did not have any effect on the NO release characteristics of either racial group (see FIGS. 4 and 5). This data reveals a fundamental difference in endothelial-dependent NO bioavailability in Mexican Americans, possibly signaling a reason why this racial group appears to be more at risk to developing cardiovascular disease. Further, this data also shows that treatment with nebivolol has the unexpected result of disproportionately increasing bioavailable NO levels in Mexican Americans to minimize or eliminate the inherent racial differences in endothelial function between this racial group and non-Hispanic Whites.

Nebivolol demonstrates highly favorable effects on the mechanisms of NO release from the endothelium of Mexican-Americans specifically, but Hispanic population in general, which can have broad implications in the treatment and/or prevention of diseases caused by endothelial-related dysfunction in this racial group.

The present invention provides for treatment and/or prevention of cardiovascular diseases associated with endothelial dysfunction, or decreased NO production, particularly in Mexican Americans and others of Hispanic descent. These disorders include, but are not limited to, hypertension, diabetes, dyslipidemia, heart failure, coronary artery disease, ischemic disease and atherosclerosis. Thus, treatment by nebivolol provides a novel therapeutic approach to such diseases, such as cardiovascular diseases, in persons of Hispanic descent, particularly Mexican Americans.

The treatment with nebivolol disclosed herein, is not concerned with the presence or identification of specific polymorphisms affecting NO release or responsiveness to nebivolol. Nebivolol was shown to improve left ventricular hypertrophy and endothelial dysfunction in subjects carrying a specific eNOS gene polymorphism, but not in those carrying another type of genetic polymorphism (Hispanic Whites). Metabolism. 45(7): 876-881, 1996. Hoffmann I S, Tavares-Mordwinkin R, Castejon A M, Alfieri A B, Cubeddu L X. Endothelial nitric oxide synthase polymorphism, nitric oxide production, salt sensitivity and cardiovascular risk factors in Hispanics. J Human Hypertens. 19: 233-240, 2005; Karimova I A, Eliseeva M R, Karimova B Sh, Abdullaeva G Zh, Adylov B Sh. Antiremodeling Activity of Nebivolol in Patients With Essential Hypertension and Various Types of 4a/4b Polymorphisms of Endothelial NO Synthase Gene, Kardiologiia. 2004; 44(8):67-71.

Nebivolol usage in Hispanics, and more specifically Mexican-Americans, can be beneficial in both treatment and prevention of endothelial dysfunction. In addition, nebivolol administration may help in the treatment of vascular disorders associated with diabetes in the Hispanic population, more specifically Mexican-Americans, who are prone to such disease.

In one aspect of the present invention, a composition comprising nebivolol is administered to a person of Hispanic descent, particularly a Mexican American, wherein nebivolol is administered in a mixture containing a greater proportion (enantiomeric excess) of the l-steroisomer of nebivolol. In another embodiment, all or substantially all of the mixture comprises the l-stereoisomer of nebivolol. NO release levels from arteries isolated from control Wistar-Kyoto (WKY) rats following treatment with nebivolol racemate, l-nebivolol or d-nebivolol vary. The order of activity (the ability to stimulate NO release) has been found to be l-nebivolol>nebivolol racemate>>d-nebivolol. It is of particular interest that whereas d-nebivolol is the active enantiomer for β₁-selective blockade, it is the weakest stimulant of NO release from WKY arterial vessels. Maximum NO release by the compounds was achieved at a concentration of 10 μM. At this concentration, the amount of NO released was 340±30 nM, 284±24 nM, and 180±21 nM for l-nebivolol, nebivolol racemate and d-nebivolol, respectively. These findings confirm that nebivolol can stimulate NO release in the intact artery in a stereoselective manner. See also, Xhonneux R, Wouters L, Reneman R S, et al. The l-enantiomer of nebivolol potentiates the blood pressure lowering effect of the d-enantiomer. Eur. J. Pharmacol. 1990; 181:261-265.

In yet another embodiment, nebivolol may be administered as a composition comprising nebivolol and one or more other active agent, particularly a cardiovascular agent, within a single dosage form, or in combination with a separately administered active agent. For example, patients of Hispanic descent, particularly Mexican Americans, may be administered a composition comprising nebivolol and one or more cardiovascular agents for the treatment and/or prevention of cardiovascular diseases. Such combinations are described in, for example, co-pending U.S. Published Application Number 2005/0272810, Davis, et al., entitled “Compositions Comprising Nebivolol,” filed May 31, 2005, and U.S. Ser. No. 11/273,992, filed Nov. 15, 2005. Hydroxylated and glucuronidated nebivolol metabolites, alone or in combination with other active agents, may also be administered to patients of Hispanic descent. To this end, US2007/0014733 “Glucuronidated nebivolol metabolites” and US2007/0014734 “Hydroxylated nebivolol metabolites” describing such metabolites and related uses, are hereby incorporated in their entirety by reference.

For example, suitable agents include a cardiovascular agent selected from the group consisting of ACE (angiotensin II converting enzyme) inhibitors, ARB's (angiotensin II receptor antagonists), adrenergic blockers, adrenergic agonists, agents for pheochromocytoma, antiarrhythmics, antiplatelet agents, anticoagulants, antihypertensives, antilipemic agents, antidiabetics, antiinflammatory agents, calcium channel blockers, CETP inhibitors, COX-2 inhibitors, direct thrombin inhibitors, diuretics, endothelin receptor antagonists, HMG Co-A reductase inhibitors, inotropic agents, rennin inhibitors, vasodialators, vasopressors, AGE crosslink breakers (advanced glycosylation end-product crosslink breakers, such as alagebrium, see U.S. Pat. No. 6,458,819), AGE formation inhibitors (advanced glycosylation end-product formation inhibitors, such as pimagedine), and mixtures thereof. In one embodiment, a cardiovascular agent such as an ACE inhibitor may be administered in combination with nebivolol to improve endothelial NO release. The ACE inhibitor may be selected from the group consisting of alacepril, benazepril, captopril, ceronapril, cilazapril, delapril, enalapril, enalaprilat, fosinopril, imidapril, lisinopril, perindopril, quinapril, ramipril, ramiprilat, spirapril, temocapril, trandolapril, and mixtures thereof. This combination of nebivolol and ACE inhibitor may then be administered to a person of Hispanic descent, particularly a Mexican American to prevent and/or treat cardiovascular disease.

In yet another embodiment, nebivolol may be administered as a composition comprising nebivolol and one or more other active agent in a single dosage form or concurrently as separate dosage forms, wherein the one or more active agent comprises a calcium channel blocker of a class selected from the group consisting of diyhdropyridines, phenylalkylamines, and benzothiazepines, wherein co-administration of the nebivolol and the one or more active agent potentiates the effect of the active agent.

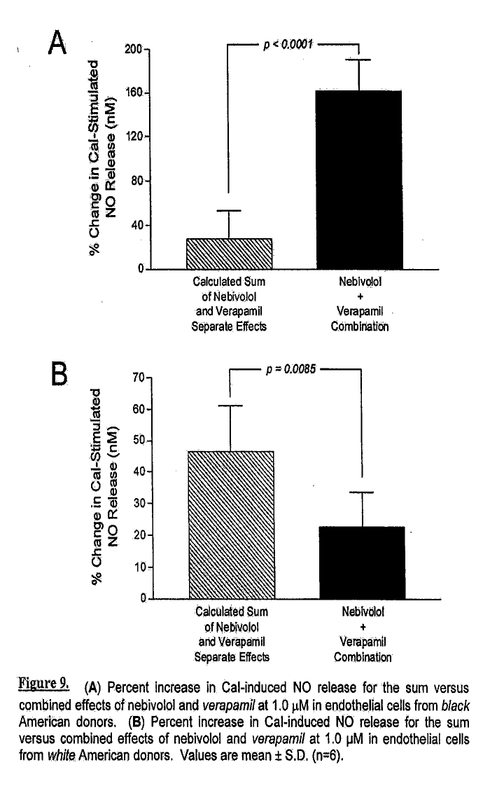

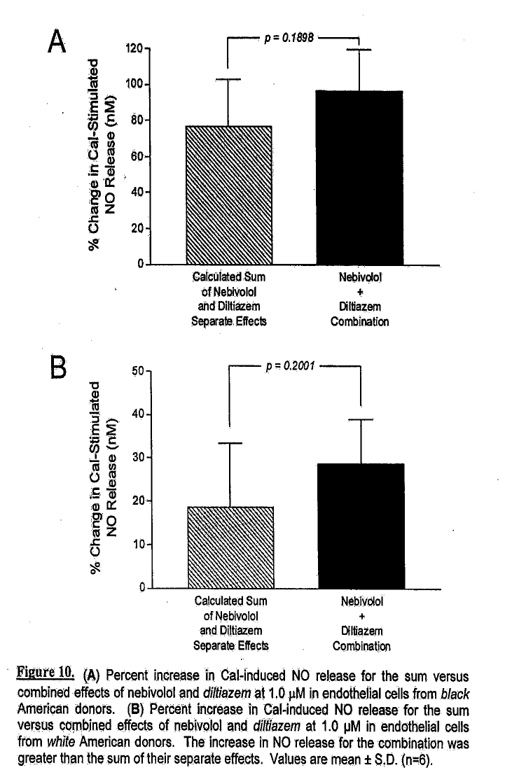

The ability for nebivolol to potentiate the effects of calcium channel blockers are shown in FIGS. 8 and 9, and 10 depicting the effects of nebivolol in combination with israpidine, verapimil and diltiazem, respectively. Nebivolol and isradipine, when administered together, demonstrates a synergistic effect in both white and black American endothelial cells. In contrast, the combination of verapimil and nebivolol exerted a synergistic effect in endothelial cells derived from black American donors, but not those from white American donors. These studies show that the combination of nebivolol and these agents result in a synergistic effect in increasing NO release that is race-dependent. As such, nebivolol treatment in combination with certain calcium-channel blockers can cause enhanced stimulation of nitric oxide that is more than additive, but rather, synergistic.

Dihydropyridine calcium channel blockers include, but are not limited to, Amlodipine (Norvasc), Felodipine (Plendil), Isradipine (DynaCirc), Nicardipine (Cardene, Carden SR), Nifedipine (Procardia, Adalat), Nimodipine Nimotop), Nisoldipine (Sular), Nitrendipine (Cardif, Nitrepin), Lacidipine (Motens), Lercanidipine (Zanidip). Phenylalkylamine calcium channel blockers include, but are not limited to, Verapamil (Calan, Isoptin) and Gallopamil (D600). Benzothiazepine calcium channel blockers include Diltiazem (Cardizem). In one particular embodiment, isradipine, a highly selective dihydropiridine calcium channel blocker, is administered in combination with nebivolol to a person of Hispanic descent, particularly a Mexican American, to treat or prevent cardiovascular disease.

### Formulation

The nebivolol compositions of the present invention may be administered by various means, depending on their intended use, as is well-known in the art. For example, if compositions of the present invention are to be administered orally, they may be formulated as tablets, capsules, granules, powders, suspensions or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations used in the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may optionally be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose will vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragées, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragées, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsion, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty. Suspensions, in addition to the subject composition, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound(s). A non-aqueous (e.g., fluorocarbon propellant) suspension could be used. Sonic nebulizers may be used because they minimize exposing the agent to shear, which may result in degradation of the compounds contained in the compositions described herein.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Pharmaceutical formulations may also be extended or delayed release formulations where the active agents are released over an extended period of time.

Dosages

Administration of the compositions of the present invention will be in an amount sufficient to achieve a therapeutic effect as recognized by one of ordinary skill in the art.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

It will be appreciated that the precise therapeutic dose of the active ingredient will depend in the age and condition of the patient and the nature of the condition to be treated and will be at the ultimate discretion of the attendant physician. However, in general effective doses for the treatment of conditions associated with coronary disorders and hypertension, will lie in the range of about 0.1 to about 50 mg, or about 1 to about 10 mg, for example about 5 mg of the active ingredient per unit dose which could be administered in single or divided doses, for example, 1 to 4 times per day. The pharmaceutical composition comprises an amount of nebivolol in the range of between about 0.125 mg and about 40 mg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

In general, the doses of an active agent will be chosen by a physician based on the age, physical condition, weight and other factors known in the medical arts.

Efficacy of Treatment

The efficacy of treatment with the compositions defined herein may be determined in a number of fashions known to those of skill in the art.

In one exemplary method, the median rate of decrease in inflammation for treatment with a subject composition may be compared to other forms of treatment with the particular cardiovascular agent contained in the subject composition, or with other cardiovascular agents. The decrease in inflammation for treatment with a subject composition as compared to treatment with another method may be 10, 25, 50, 75, 100, 150, 200, 300, 400% greater or even more. The period of time for observing any such decrease may be about 1, 3, 5, 10, 15, 30, 60 or 90 or more hours. The comparison may be made against treatment with the particular cardiovascular agent contained in the subject composition, or with other cardiovascular agents, or administration of the same or different agents by a different method, or administration as part of a different drug delivery device than a subject composition. The comparison may be made against the same or a different effective dosage of the various agents.

Alternatively, a comparison of the different treatment regimens described above may be based on the effectiveness of the treatment, using standard indices known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different cardiovascular agents.

Kits

This invention also provides kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### EXEMPLIFICATION

### Example 1

The ability of nebivolol to enhance NO bioavailability in endothelial cells from non-Hispanic white and Mexican American donors, as compared to atenolol was tested. The effects of nebivolol and atenolol on endothelial-dependent NO release were tested in human umbilical vein endothelial cells (HUVECs). Human umbilical vein endothelial cells were isolated into primary cultures from non-Hispanic white and Mexican American female donors by Clonetics (San Diego, Calif.) and purchased as proliferating cells. All cell culture donors were healthy, with no pregnancy or prenatal complications. None of the donors took any drugs regularly and all were nonsmokers and consumed regular caloric/content diet. The identification of race was defined by self-report.

The cultured cells were incubated in 95% air/5% CO₂ at 37° C. and passage by an enzymatic (trypsin) procedure. The confluent cells (4 to 5×10⁵ cells/35-mm dish) were placed with minimum essential medium containing 3 mmol/L L-arginine and 0.1 mmol/L BH4 [(6R)-5,6,7,8-tetrahydrobiopterin]. Before the experiments, the cells (from second or third passage) were rinsed twice with Tyrode-HEPES buffer with 1.8 mmol/L CaCl₂. Drugs were obtained from Mylan Laboratories (Morgantown, W. Va.).

NO measurement was carried out with electrochemical nanosensors The design was based on previously developed and well-characterized chemically modified carbon-fiber technology (Malinski, Nature 358, 676-678, 1992; Lvovich, V., Scheeline, A. Anal. Chem. 69, 454-462, 1997). Each of the nanosensors was made by depositing a sensing material on the tip of a carbon fiber (length 4-5 μm, diameter 0.2-0.5 μm). The fibers were sealed with nonconductive epoxy and electrically connected to copper wires with conductive silver epoxy. We used a conductive film of polymeric nickel (II) tetrakis (3-methoxy-4-hydroxyphenyl) porphyrin for the NO-sensor.

The NO nanosensors (diameter 1-2 μm) with a platinum wire (0.1 mm) counter electrode and saturated calomel reference electrode (SCE) were applied. Differential pulse voltammetry (DPV) and amperometry were performed with a computer-based Gamry VFP600 multichannel potentiostat. DPV was used to measure the basal NO concentrations, and amperometry was used to measure changes in NO concentrations from its basal level with time. The DPV current at the peak potential characteristic for NO oxidation (0.65 V) reduction was directly proportional to the local concentrations of these compounds in the immediate vicinity of the sensor. Linear calibration curves (current vs. concentration) were constructed for each sensor from 10 nmol/L to 3 μmol/L before and after measurements with aliquots of NO standard solutions, respectively. The detection limit of the sensors was 10⁻⁹ mol/L. The quantification of each analyte (concentration in nmol/L) was performed using a maximum current from amperograms and standard calibration curves. A reproducibility of measurements with nanosensors is relatively high (between 5-12%). The NO nanosensor modules were lowered with the help of a computer-controlled micromanipulator until it reached the surface of the cell membrane (a small piezoelectric signal, 0.1-0.2 pA, of 1-3 milliseconds duration was observed at this point). The sensors were slowly raised 4±1 μm from the surface of a single endothelial cell.

The HUVEC preparation is stable over the course of these experiments with the cells remaining viable in culture for >24 hours. Under non-stimulating conditions, basal levels of NO release are very low (<30 nM). Multiple measurements of NO release were conducted on single cells following a brief refractory period. For robust statistical analysis, separate cells were used for each concentration and type of drug used in these analyses.

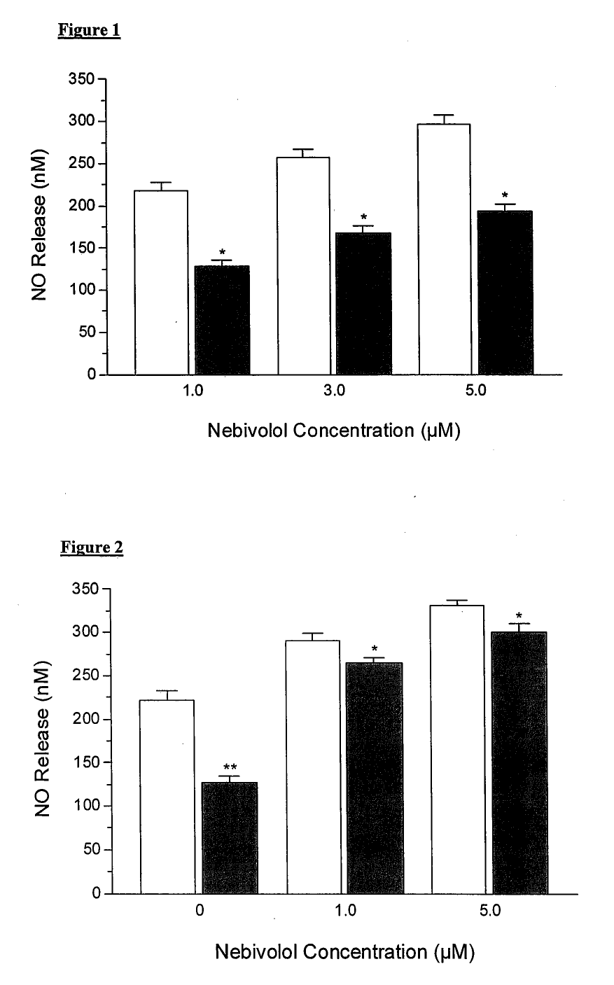

These experiments demonstrate a dramatic difference in endothelial-dependent NO release in HUVECs obtained from non-Hispanic white versus Mexican American donors following acute administration of nebivolol over a range of concentrations (1.0-5.0 μM) (FIG. 1). As compared to the non-Hispanic white donors, release of NO from Mexican American endothelium was 43% less following stimulation with the agonist. Consistent with previous studies, acute treatment with nebivolol can cause endothelial-dependent NO release. However, to enhance the ability of the cells to generate NO, longer-term treatment is necessary. Indeed, as shown in FIGS. 2 and 3, the effects of 24 h pretreatment with nebivolol at two different concentrations (1.0 and 5.0 μM, respectively) produced dramatic differences in the ability of the cells to generate NO, especially in tissue from the Mexican American donors. While nebivolol pretreatment caused an increase in NO bioavailability in cells from both groups of donors, the benefit was disproportionately greater in tissue from Mexican Americans. This is consistent with what had been observed in the endothelium from African American subjects, though the mechanism may be distinct.

The increases in NO release following 24 h pretreatment with nebivolol at two different concentrations are shown in FIGS. 4 and 5. This analysis demonstrates the remarkable and disproportionate benefit with nebivolol in endothelium from Mexican American donors, as compared to white donors. The increase in NO bioavailability with nebivolol at 1.0 μM was two-fold greater in Mexican American samples; nebivolol caused an increase of 68±3 nM in cells from white donors and 138±2 nM in Mexican American donor cells (FIG. 4). In contrast, the addition of atenolol failed to stimulate any significant amount of NO release from either racial group under identical conditions. This data, too, is consistent with previous findings in African American subjects, though the underlying mechanism is undetermined.

Collectively, these data demonstrate fundamental differences in endothelial-dependent NO bioavailability in Mexican Americans, a factor that may contribute to the increased risk for CV disease and reduced blood pressure control in this population, as compared to the U.S. population as a whole. Remarkably, pretreatment with nebivolol essentially eliminated racial differences in endothelial function, and enhanced overall NO release in both non-Hispanic whites and Hispanic American, especially Mexican American donors. These data provide the first evidence for a direct and disproportionate endothelial benefit with nebivolol in this rapidly growing segment of the Hispanic American population, independently of β₁-blockade.

### Example II

Endothelial function in cells from normotensive, age-matched Mexican American (MA) and Non-Hispanic White (NHW) donors, as well as the effects of treatment with nebivolol was also determined. Endothelial NO and peroxynitrite (ONOO⁻) release was measured simultaneously in human umbilical vein endothelial cells from age-matched MA and NHW donors using a nanosensor array. The effects of nebivolol on NO and ONOO⁻ production was evaluated following pre-treatment (24 h) using calcium ionophore (CaI) as a receptor-independent stimulus. Levels of eNOS were measured by Western blot analysis. Drug-membrane interactions were determined by small angle x-ray diffraction analysis. Endothelial NO bioavailability from MA donors was 30% lower than in NHW (383±10 nM versus 543±8 nM, mean±S.E.M.) following stimulation with CaI (1.0 μM). Remarkably, pretreatment (24 hr) of the cells with nebivolol (1.0 μM) eliminated these interracial differences and enhanced NO release disproportionately in MA cells (57%) versus NHW cells (20%). Nebivolol also reduced elevated ONOO⁻ levels in MA endothelium by 75% (746±12 nM to 195±10 nM) and 50% in NHW (416±7 nM to 191±13 nM). The ratio of NO to ONOO—, an indicator of eNOS coupling, increased more than 5-fold in MA following nebivolol treatment. Finally, eNOS levels were 40% lower in MA endothelium compared to NHW but increased by two-fold with nebivolol treatment. These effects were not observed with atenolol, a β₁-adrenergic receptor selective antagonist (beta-blocker) and hydrophilic agent that differs from nebivolol in its membrane location, antioxidant properties and eNOS activity. These data demonstrate clear differences between MA and NHW in endothelial NO bioavailability and nitroxidative stress—factors that may contribute to increased CV risk. Treatment with nebivolol eliminated interracial differences by enhancing both the expression and coupling efficiency of eNOS.

### EQUIVALENTS

Those skilled in the art will recognize, or be able to ascertain using no more than routine experimentation, many equivalents to the specific embodiments of the invention described herein.

1. A method of reducing morbidity or mortality associated with cardiovascular disease in a person of Hispanic descent comprising administering to the person a therapeutically effective amount of nebivolol or a pharmaceutically acceptable salt thereof.

2. The method of claim 1 wherein the cardiovascular disease is selected from the group consisting of congestive heart failure, hypertension, pulmonary hypertension, myocardial and cerebral infarctions, atherosclerosis, atherogenesis, thrombosis, ischemic heart disease, post-angioplasty restenosis, coronary artery diseases, renal failure, stable, unstable and variant (Prinzmetal) angina, cardiac edema, renal insufficiency, nephrotic edema, hepatic edema, stroke, transient ischemic attacks, cerebrovascular accidents, restenosis, controlling blood pressure in hypertension, platelet adhesion, platelet aggregation, smooth muscle cell proliferation, pulmonary edema, vascular complications associated with the use of medical devices, wounds associated with the use of medical devices, pulmonary thromboembolism, cerebral thromboembolism, thrombophlebitis, thrombocytopenia and bleeding disorders.

3. The method of claim 1, wherein the cardiovascular disorder is hypertension.

4. The method of claim 1 wherein the nebivolol is administered in an amount of from about 1 mg per day to about 40 mg per day.

5. A method of claim 1, wherein the person of Hispanic descent is a Mexican American.

6. A method for improving NO release in a person of Hispanic descent in need thereof, comprising administering to the person a safe and therapeutically effective amount of nebivolol or a pharmaceutically acceptable salt thereof.

7. The method according to claim 6 wherein nebivolol or a pharmaceutically acceptable salt is administered in an amount of about 1 mg per day to about 40 mg per day.

8. A method according to claim 6, wherein the person of Hispanic descent is a Mexican American.

9. A method for improving exercise tolerance or for improving the quality of life in a person of Hispanic descent in need thereof, comprising administering to the person a therapeutically effective amount of nebivolol or a pharmaceutically acceptable salt thereof.

10. A method according to claim 9 wherein nebivolol or a pharmaceutically acceptable salt thereof is administered in the amount of about 1 mg to about 40 mg per day.

11. A method according to claim 9, wherein the person of Hispanic descent is a Mexican American.

12. A method of treating and/or preventing a cardiovascular condition in a person of Hispanic descent in need thereof, comprising administering to a subject a therapeutically safe and effective amount of nebivolol or pharmaceutically acceptable salt and at least one other active agent, sufficient to treat said condition.

13. The method of claim 12 wherein the at least one other active agent is a cardiovascular agent.

14. The method of claim 13 wherein the cardiovascular agent is selected from the group consisting of ACE inhibitors, ARB's, adrenergic blockers, adrenergic agonists, agents for pheochromocytoma, anti-arrhythmics, antiplatelet agents, anticoagulants, antihypertensives, antilipemic agents, antidiabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, renin inhibitors, vasodilators, vasopressors, AGE crosslink breakers, AGE formation inhibitors, and mixtures thereof.

15. The method of claim 13 wherein the cardiovascular condition is selected from the group consisting of congestive heart failure, hypertension, pulmonary hypertension, myocardial and cerebral infarctions, atherosclerosis, atherogenesis, thrombosis, ischemic heart disease, post-angioplasty restenosis, coronary artery diseases, renal failure, stable, unstable and variant (Prinzmetal) angina, cardiac edema, renal insufficiency, nephrotic edema, hepatic edema, stroke, transient ischemic attacks, cerebrovascular accidents, restenosis, controlling blood pressure in hypertension, platelet adhesion, platelet aggregation, smooth muscle cell proliferation, pulmonary edema, vascular complications associated with the use of medical devices, wounds associated with the use of medical devices, pulmonary thromboembolism, cerebral thromboembolism, thrombophlebitis, thrombocytopenia and bleeding disorders.

16. The method of claim 13 wherein the cardiovascular agent is selected from the group consisting of ACE inhibitors, ARB's, and mixtures thereof.

17. The method of claim 13 wherein the cardiovascular agent is an ACE inhibitor.

18. The method of claim 13 wherein the cardiovascular agent is an ARB.

19. The method of claim 13 wherein the cardiovascular agent is selected from the group consisting of AGE crosslink breakers, AGE formation inhibitors, and mixtures thereof.

20. The method of claim 13 wherein the cardiovascular agent is a calcium channel blocker.

21. A method of claim 12, wherein the person of Hispanic descent is Mexican American.
